(19) United States
(12) Patent Application Publication
(10) Pub. No.: US 2024/0382564 A1
(43) Pub. Date: Nov. 21, 2024

(54) Methods and Compositions for Treating Muscular Dystrophy and Memory Impairment

(71) Applicant: Allysta Pharmaceuticals, Inc., Bellevue, WA (US)

(72) Inventor: Henry Hsu, Bellevue, WA (US)

(21) Appl. No.: 18/637,450

(22) Filed: Apr. 17, 2024

Related U.S. Application Data
(60) Provisional application No. 63/496,639, filed on Apr. 17, 2023.

(51) Int. Cl. A61K 38/22; A61P 9/00; A61P 21/00; A61P 25/28

(52) U.S. Cl. CPC A61K 38/22; A61P 9/00; A61P 21/00; A61P 25/28

(57) ABSTRACT

A method of treating a muscular dystrophinopathy, or a condition associated therewith, in a subject by administering to the subject a therapeutically effective amount of a composition comprising ALY688. The muscular dystrophinopathy can be Duchenne muscular dystrophy (DMD), Becker muscular dystrophy (BMD), among others.

### INCORPORATION Of SEQUENCE LISTING

This application includes a Sequence Listing which has been submitted in XML format via Patent Center, named “ALLY008.XML” which is 10 KB in size and created on Apr. 3, 2024. The contents of the Sequence Listing are incorporated herein by reference in their entirety.

### BACKGROUND

Duchenne muscular dystrophy (DMD) is a progressive inherited neuromuscular disorder occurring in approximately 1 in every 5,000 live male births. Progressive striated (skeletal and cardiac) muscle weakness from DMD in childhood due to genetic alterations in dystrophin usually leads to an early dependence on assistive devices as well as a reduced lifespan from respiratory or cardiac failure. A loss of cell membrane stability in striated muscles renders these cells susceptible to contraction-induced damage prompting them to undergo repeated cycles of necrosis and regeneration until muscle mass is progressively replaced by fibrous connective tissue and fat resulting in progressive muscle weakness and loss of function.

People with DMD may also exhibit cognitive dysfunction including memory impairments. Such cognitive impairments may be related to disruptions in brain energy homeostasis. The effects of GC on memory in DMD is not fully resolved in the literature, but memory impairments attributed to GC therapy have been identified more broadly in other populations.

Glucocorticoids (GC) are the current standard of care for slowing the progression of muscle weakness in males with DMD by reducing inflammation. Chronic GC use is associated with many adverse effects including excessive weight gain, adrenal insufficiency, stunted growth, cushingoid appearance, behavioural changes, decreased bone mineral density and increased incidence of fractures,.

As such, there is an unmet need for new therapies that ameliorate the muscle damage without the severe adverse effects seen with GC.

### DETAILED DESCRIPTION

The term “peptide” refers to an organic compound comprising a chain of two or more amino acids covalently joined by peptide bonds. Peptides may be referred to with respect to the number of constituent amino acids, i.e., a dipeptide contains two amino acid residues, a tripeptide contains three, etc. A peptide may be a backbone modified peptide, any polyamide or other polymeric structure resembling peptides, peptides containing non-natural amino acid residues or a peptide derivative.

It has been now discovered that the peptide DAsn-Ile-Pro-Nva-Leu-Tyr-DSer-Phe-Ala-DSer-NH₂ (SEQ ID NO:1), also referred to as ALY688 herein, can induce a wide range of actions within the cell eventually resulting in preservation of muscle function, as well as inhibition of inflammation and fibrosis in muscles. “Nva” in ALY688 corresponds to the non-natural amino acid norvaline, also known as 2(L)-aminopentanoic acid.

The term “therapeutically effective amount,” “effective amount” or “therapeutically effective dose” refers to that amount of the therapeutic agent or composition sufficient to ameliorate a disorder, as described above. For example, for the given parameter, a therapeutically effective amount will show an increase or decrease of therapeutic effect at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control. In the context of the present invention, the effective amount of an adiponectin peptidomimetic compound can vary depending on co-administration of other therapeutics or disease profile of the individual (among other factors such as age, severity of disease, etc.).

The term “treat”, “treating”, or “treatment” refers to the treating or treatment of a disease or medical condition in a patient, such as a mammal (particularly a human or an animal) which includes: ameliorating the disease or medical condition, i.e., eliminating or causing regression of the disease or medical condition in a patient; suppressing the disease or medical condition, i.e., slowing or arresting the development of the disease or medical condition in a patient; or alleviating one or more symptoms of the disease or medical condition in a patient. The term encompasses the prophylactic treatment of a disease or condition as to prevent or reduce the risk of acquiring or developing a specific disease or condition, or to prevent or reduce the risk of recurrence.

The term “subject,” “individual” or “patient” typically includes humans, but can also include other animals such as, e.g., other primates, rodents, canines, felines, equines, ovines, porcines, and the like.

The word “about” as used herein in association with a numeric value or a numeric range mean “approximately” and refers to a result that can be obtained within a tolerance and the skilled person knows how to obtain the tolerance, for example, ±20% of the given value or range.

### Preparations of Compounds of the Formulation of the Invention

Free form peptide compounds of the present invention may be recombinant peptides or synthetic peptides. They may also be chemically synthesized, using, for example, solid phase synthesis methods. Additionally, peptide transduction domains appended to peptides of the invention may be natural or synthetic peptides, and may be either prepared by isolation from natural sources or may be synthesized.

The peptides of the present invention may be synthesized de novo using peptide synthesis methods. In such methods, the peptide chain is prepared by a series of coupling reactions in which the constituent amino acids are added to the growing peptide chain in the desired sequence. The use of various N-protecting groups, e.g., the carbobenzyloxy group or the t-butyloxycarbonyl group; various coupling reagents e.g., dicyclohexylcarbodiimide or carbonyldiimidazole; various active esters, e.g., esters ofFloss N-hydroxyphthalimide or N-hydroxy-succinimide; and the various cleavage reagents, e.g., trifluoroacetic acid (TFA), HCl in dioxane, boron tris-(trifluoracetate) and cyanogen bromide; and reaction in solution with isolation and purification of intermediates are methods well-known to those of ordinary skill in the art. The reaction may be carried out with the peptide either in solution or attached to a solid phase support. In the solid phase method, the peptide is released from the solid phase support following completion of the synthesis.

In some embodiments, the peptide synthesis method may follow Merrifield solid-phase procedures. See, e.g., Merrifield, J. Am. Chem. Soc, 1963, 85, 2149-54. Additional information about the solid phase synthetic procedure can be obtained from, for example, Solid Phase Peptide Synthesis: A Practical Approach by E. Atherton and R. C. Sheppard (Oxford University Press, 1989, Solid phase peptide synthesis, by J. M. Stewart and J. D. Young, (2nd edition, Pierce Chemical Company, Rockford, 1984), and the review chapters by R. Merrifield in Advances in Enzymology 32:221-296, edited by F. F. Nold (Interscience Publishers, New York, 1969) and by B. W. Erickson and R. Merrifield in The Proteins Vol. 2, pp. 255 et seq., edited by Neurath and Hill, (Academic Press, New York, 1976). Peptide synthesis may follow synthetic techniques such as those set forth in Fields et al., Introduction to Peptide Synthesis, in Current Protocols in Molecular Biology (Chapter 11, Unit 11.15; John Wiley and Sons, 2008) and Amblard et al. (2006, Molecular Biotechnology, 33:239-254).

The synthesis of peptides by solution methods is described in, for example, The Proteins, Vol. 11, edited by Neurath et al. (3rd Edition, Academic Press 1976). Other general references to the synthesis of peptides include: Peptide Synthesis Protocols, edited by M. W. Pennington and Ben M. Dunn (Humana Press 1994), Principles of Peptide Synthesis, by Miklos Bodanszky (2nd edition, Springer-Verlag, 1993), and Chemical Approaches to the Synthesis of Peptides and Proteins by Paul Lloyd-Williams, F. Albericio, E. Giralt (CRC Press 1997), and Synthetic Peptides: A User's Guide, edited by G. Grant (Oxford University Press, 2002).

The nucleic acid encoding a desired peptide may be operatively linked to one or more regulatory regions. Regulatory regions include promoters, polyadenylation signals, translation initiation signals (Kozak regions), termination codons, peptide cleavage sites, and enhancers. The regulatory sequences used must be functional within the cells of the vertebrate in which they are administered. Selection of the appropriate regulatory region or regions is a routine matter, within the level of ordinary skill in the art.

The compounds of the invention, whether prepared by chemical synthesis or recombinant DNA technology, may be purified using known techniques, for example preparative HPLC, FPLC, affinity chromatography, as well as other chromatographic methods. Isolated compounds may then be assessed for biological activity according to the methods described herein, as well as by any methods known to the skilled artisan.

### ALY688 Compositions/Formulations

ALY688 can be prepared for use in the form of a pharmaceutical composition, in combination with a pharmaceutically acceptable carrier. “Pharmaceutically acceptable carrier” means any carrier, diluent or excipient which is compatible with the other ingredients of the formulation and not deleterious to the recipient. The carrier can be selected on the basis of the selected route of administration and standard pharmaceutical practice.

ALY688 can be provided in pharmaceutical compositions suitable for administration by various routes, e.g., by subcutaneous or other parental routes. Salt of ALY688, such as ALY688 acetate or ALY688-deoxycholate can be used in the composition. A pH adjusting agent, such as hydrochloric acid, NaH₂PO₄, or a pH buffer system, can also be used, such that the pH of the composition can be adjusted as appropriate. Stabilizing agents, antioxidant agents and preservatives may also be added. The composition for parenteral administration may take the form of an aqueous solution, dispersion, suspension or emulsion. ALY688 can constitute from about 0.01 wt % to about 20 wt % of the total composition. It is to be noted that except those explicitly stated agents or components, the balance of any aqueous solution/suspension of ALY688 described herein is water.

The term “ALY688-deoxycholate”, or “ALY688-DC” refers to the ALY688 deoxycholate salt, which comprises equal molar amounts of the positively charged ALY688 and a deoxycholate anion as an inactive constituent. In some embodiments, the disclosure provides a composition comprising ALY688-DC. In examples of this disclosure, ALY688 composition used includes the form of ALY688-DC.

In some embodiments, the ALY688 composition can use other salts such as acetate, chloride, phosphate, lipidic salts.

In some embodiments, the ALY688 composition can include non-ionic surfactants, such as a polyoxyethylene sorbitan monofatty acid ester having 12 to 18 carbon atoms, a sorbitan fatty acid ester having 16 to 18 carbon atoms, glycerol monooleate, glycerol dilaurate, glycerol distearate, glycerol dioleate, polyoxyethylene castor oil, etc. In some embodiments, the non-ionic surfactant can include polysorbate 20 (also known as TWEEN 20).

In some embodiments, the ALY688 composition herein can include Polyethylene Glycol (PEG) at different molecular weights, e.g. PEG3350, polyvinyl pyrrolidone (PVP), Poloxamers, mannitol, trehalose and sucrose.

### Methods of Treatment using ALY688 formulations

The ALY688 formulations described herein can be used to treat a condition or disease of a subject. Thus, in another aspect, the present disclosure provides a method for treating a disease or condition in a subject, e.g., a human or non-human mammal, comprising administering to the subject a therapeutically effective amount of the pharmaceutical composition described herein.

The diseases that can be treated by the compositions of the present disclosure include a muscular dystrophinopathy or a symptom/condition associated therewith. The muscular dystrophinopathy can be one of Duchenne muscular dystrophy (DMD), Becker muscular dystrophy (BMD), DMD-associated dilated cardiomyopathy (DCM), facioscapulohumeral muscular dystrophy (FSHD), congenital muscular dystrophy (CMD), Emery-Dreiffus muscular dystrophy (EDMD), Limb-girdle muscular dystrophy (LGMD), myotonic dystrophy, and oculopharyngeal muscular dystrophy (OPMD). In some embodiments, the condition is loss of muscle and function in the right ventricle. In some embodiments, the condition is diaphragm atrophy. In some embodiments, the symptom is memory impairment.

The pharmaceutical composition can be suitable for intramuscular, subcutaneous, parenteral, epidermal, and other routes of administration. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the invention can be administered via a non-parenteral route, such as a topical, epidermal or mucosal route of administration, e.g., intranasally, orally, vaginally, rectally, sublingually or topically. In particular embodiments, the administration is through subcutaneous route.

In the administration of the composition to the subject, dosage regimens can be adjusted to provide the optimum desired response (e.g., a therapeutic response). Single bolus or divided doses can be administered based on the subject, the disease to be treated, etc. Dosage unit form as used herein refers to physically discrete units suited as unitary dosages for the subjects to be treated. Each unit contains a predetermined quantity of active ingredient calculated to produce the desired therapeutic effect in association with the required pharmaceutical carrier. Sustained release formulation can be used in which case less frequent administration is required.

For administration of the aqueous composition of the present disclosure, the dosage may range from about 0.0001 to 100 mg/kg, and more usually 0.01 to 5 mg/kg, of the body weight of the subject. For example dosages can be 0.3 mg/kg body weight, 1 mg/kg body weight, 3 mg/kg body weight, 5 mg/kg body weight or 10 mg/kg body weight or within the range of 1-10 mg/kg. A suitable treatment regime can be once per day, twice per day, once every two days, once every three three, once every week, once every other week, once a month, etc. Example dosage regimens for an aqueous composition of the invention can include 1 mg/kg body weight or 3 mg/kg body weight via subcutaneous administration.

The Male D2.mdx mouse is a genetically modified mouse that contains the same defect in dystrophin production seen in the human condition. As such, these animals develop many of the same features of progressive muscle damage seen in humans and are a useful model to study the effects of therapies.

### Example 1: Mitigation of Diaphragm Fibrosis, Atrophy, and Mitochondrial Stress in a Duchenne Muscular Dystrophy Mouse Model

### Methods

### Animal Care

Male D2.mdx mice were bred from an in-house colony established at York University (Toronto, Ontario) and treated from 7 to approximately 28 days of age. 3 week-old DBA/2J wildtype (WT) mice were ordered directly from Jackson Laboratories (Bar Harbor, USA) due to low breeding performance experienced in-house and allowed to acclimatize for 7 days prior to sacrifice. All experiments and procedures were approved by the Animal Care Committee at York University (AUP Approval Number 2016-18) in accordance with the Canadian Council on Animal Care.

### ALY688 Treatment

See Supporting Information for more details.

### ALY688 Treatment

D2.mdx mice were treated with ALY688 at two different dosages; high dose (HD; 15 mg/kg body weight/day) or low dose (LD; 3 mg/kg body weight/day) dissolved in saline. A separate group of mice were treated with saline alone (VEH, vehicle). Treatment was provided via subscapular subcutaneous injections from 7 days of age to 28-30 days. Wildtype mice did not receive treatment. At 28-30 days of age, mice were anaesthetized with isoflurane vaporized in medical air (21% oxygen) at a 2 L/min flow rate. Due to tissue limitations in limb muscle, certain measures were performed separately in tibialis anterior and quadriceps across several phases of breeding. Following the treatment protocol, in situ quadriceps force assessments were then performed in one limb followed by removal of the quadriceps from the other limb for mitochondrial assessments, diaphragm for in vitro force assessments and finally other tissues for further processing as described below. Muscle and visceral organs weights were normalized to tibia length. Tissue collected from the first phase were utilized in force, histological, mitochondrial measures (respiration, mH₂O₂ and calcium retention capacity) and western analyses (diaphragm and quadriceps only). A second phase was then collected and utilized for cytokine profiling, qPCR analyses and additional westerns (TA only). Serum was collected from both phase 1 and 2 and utilized for serum CK analysis. All animals were utilized in in vivo functional assays (grip strength, cage hang time and voluntary wheel running).

### Histochemical Analysis of Muscle Regeneration

Centralized nuclei, a marker of regenerating fibres, was analyzed by selecting five evenly spaced images throughout the muscle and averaging the result.

### RNA Isolation & Quantitative PCR

Samples were lysed using TRIzol reagent and RNA was separated to aqueous phase using chloroform. The aqueous layer containing RNA was then mixed with isopropanol and loaded to Aurum Total RNA Mini Kit columns (Bio-Rad, Mississauga, ON, Canada). Total RNA was then extracted according to the manufacturer's instructions and concentration was assessed on the nanodrop attachment for the Varioskan LUX Multimode Microplate reader (Thermo Scientific). Reverse transcription of RNA into cDNA was performed by M-MLV reverse transcriptase and oligo (dT) primers (Qiagen). cDNA was then amplified in a CFX384 Touch Real-Time PCR Detection Systems (Bio-Rad) with a SYBR Green master mix and specific primers. Standard curves were created by plotting log DNA concentrations against Ct values from a 1:5 serial dilutions with CFX Manager Software. Gene expression was normalized to a Rplp0 control, and relative differences were determined using the ΔΔCt method Values are presented as fold increases relative to WT group.

### Cytokine Profiling

Tibialis anterior and diaphragm samples were homogenized in lysis buffer containing (in mM) (20 Tris/HCl, 150 NaCl, 1 EDTA, 1 EGTA, 1% Triton X-100, 2.5 Na₄O₇P₂, 1 Na₃VO₄, pH 7.0) supplemented with protease (Roche) and phosphatase inhibitors (Sigma). Samples were diluted two-fold in the assay buffer before loading to the plates with beads. According to the manufacturer's instructions, levels of TGF-β, TNF-α, IL-1β, IL-6 and IL-10 in the homogenates were measured using LEGENDplex™ Mouse Custom Panel. Assay was performed in the Attune NxT flow cytometer (Thermo Fisher). The FCS files generated on the flow cytometer were analyzed using the LEGENDplex™ cloud-based analysis software.

### In-Vitro Diaphragm Force

A silk suture was attached to the central tendons and the ribs and secured in a bath with oxygenated Ringers solution containing (in mM) 121 NaCl, 5 KCl, 1.8 CaCl₂, 0.5 MgCl₂, 0.4 NaHPO₄, 24 NaHCO₃, 5.5 glucose and 0.1 EDTA; pH 7.3 (95% O₂/5% CO₂) maintained at 25° C. with ribs secured to the force transducer with an s-hook and central tendon to the lever arm. The diaphragm was positioned between two platinum electrodes driven by a biphasic stimulator (Model 305C; Aurora Scientific, Inc., Aurora, ON, Canada) and allowed to acclimatize for 30 minutes. Optimal resting length (L₀) was found using single twitches until maximal force was attained. A force-frequency curve was performed (1, 10, 20, 40, 60, 80, 100, 120, 140, 200 Hz) with 1 minute of rest between contractions with a final 5-minute rest period provided before a fatiguing protocol was performed (70 Hz for 350 ms every 2 seconds for 5 minutes). After the force-frequency protocol was assessed, a 5 minute rest period was provided before a fatiguing protocol was performed (70 Hz for 350 ms every 2 seconds for 5 minutes). Recovery from fatigue was assessed at 5-, 10- and 15-minutes post fatigue at the frequency that elicited maximum force in the force-frequency protocol. Force production was analyzed using Dynamic Muscle Control Data Acquisition software (Aurora Scientific, Inc) and normalized to cross sectional area (CSA) of the muscle strip (m/l*d) where ‘m’ is the mass of the strip devoid of the central tendon and ribs, ‘l’is the length (from the point of insert on the ribs to the myotendinous junction of the central tendon) and ‘d’ is the mammalian skeletal muscle density (1.06 g/mm³).

### In-Situ Quadriceps Force

Body temperature was maintained using an overhead heat lamp. Once fully sedated, hair was removed from the knee and a small vertical incision was made above the knee to expose the patellar tendon. The tendon was then secured with a suture (4/0; Fine scientific instruments, North Vancouver, Canada), with a loop at the end. The tendon was then severed, and the loop was then attached to an Aurora Scientific 305C muscle lever arm with an s-hook (Aurora, Ontario, Canada). A 27 G needle was pierced from the lateral side of the knee through the femoral epicondyles immobilizing the knee joint. The knee was then secured with a vertical knee clamp and quadriceps contraction was facilitated through percutaneous stimulation of the femoral nerve. Optimal resting length (L₀) was determined using single twitches (pulse width=0.2 ms) at varying muscle lengths at the maximal stimulation current. Force was assessed across increasing frequencies (1, 40, 60, 80, 100, 120 Hz) using a biphasic stimulator (Model 710B, Aurora Scientific, Inc., ON, Canada), with one minute rest between each frequency. After a five minute rest period, a fatigue protocol (70 Hz stimulation once every 1.5 s for 120 contractions) was initiated. Recovery from fatigue was assessed 5, 10 and 15 minutes after the last contraction of the fatigue protocol at the frequency that elicited max force. Data were analyzed using Dynamic Muscle Control Data Acquisition software (Aurora Scientific, Inc) and normalized to quadriceps weight in mg.

### Preparation of Permeabilized Muscle Fibre Bundles

The quadriceps from the non-stimulated limb and the diaphragm were collected and quickly placed in a buffer containing (in mM) 50 MES Hydrate, 7.23 K₂EGTA, 2.77 CaK₂EGTA, 20 imidazole, 0.5 dithiothreitol, 20 taurine, 5.77 ATP, 15 PCr, and 6.56 MgCl₂·6H₂O (pH 7.1). Both muscles were trimmed of connective tissue and fat and separated along the longitudinal axis into small bundles weighing approximately 1.0-2.5 mg wet weight for respiration and mH₂O₂ and 0.5-1.5 mg wet weight for calcium retention capacity. Bundles were permeabilized with 40 μg/μL saponin (Sigma Aldrich; St. Louis, MI, USA) in BIOPS on a platform rotor for 30 minutes at 4° C. The permeabilized bundles were then transferred to wash buffers to remove saponin as follows: MiRO5 for mitochondrial respiration, Buffer Z for mitochondrial H₂O₂ emission (mH₂O₂), and Buffer Y for calcium retention capacity. The composition of each buffer has been described previously. Fibres prepared for pyruvate-supported mitochondrial H₂O₂ emission (mH₂O₂) were permeabilized in the presence of 35 μM 2,4-dinitrochlorobenzene (CDNB) to deplete glutathione and enhance the detection of mH₂O₂. All bioenergetic assays were performed within 4 hours of washing to maintain fibre viability.

### Mitochondrial Respiration

High resolution oxygen consumption (an index of mitochondrial respiration) was performed in 2 mL of MiRO5 supplemented with (creatine-dependent) or without (creatine independent) 20 mM creatine to saturate mitochondrial creatine kinase activity. O₂ consumption was measured using the Oroboros Oxygraph-2K (Oroboros Instruments, Corp., Innsbruck, Austria) while stirring at 37° C. in the presence of 5 μM blebbistatin to prevent muscle fibre contraction by rigor in response to ADP. Each chamber was oxygenated with 100% pure O₂ to an initial concentration of ˜350 μM and experiments were completed before chamber [O₂] reached 150 μM. Prior to permeabilization, fibre bundles were gently and quickly blotted dry, weighed in ˜1.5 mL of tared cold BIOPS (ATP-containing relaxing media) to ensure fibres remained relaxed. Respiration was normalized to bundle wet weight. Complex I-supported respiration was stimulated using 5 mM pyruvate and 2 mM malate (NADH; State II respiration) followed by a titration of ADP concentrations (State III) from physiological ranges (25, 100 μM) to supraphysiological (500 μM) and saturating to stimulate maximal coupled respiration (5000 μM). An addition of 10 μM cytochrome c was added to test mitochondrial outer membrane integrity. Experiments with very low ADP-stimulated respiration and high cytochrome c responses were removed. Lastly, 10 mM succinate (FADH₂) was added to support complex-II respiration.

Mitochondrial H₂O₂ Emission (mH₂O₂)

Briefly, mH₂O₂ was determined spectrofluorometrically (QuantaMaster 40, HORIBA Scientific, Irvine, CA, USA) utilizing Buffer Z containing 10 μM Amplex Ultra Red (Life Technologies; Carlsbad, CA, USA), 1 U/mL horseradish peroxidase, 1 mM EGTA, 40 U/mL Cu/Zn SOD1, 5 μM blebbistatin and 20 mM Cr. Complex I-supported mH₂O₂ was initiated through the addition of 10 mM pyruvate and 2 mM malate (NADH; forward electron flow) and separately with 10 mM succinate (FADH₂; reverse electron flow from Complex II to I). The ability of ADP to suppress mH₂O₂ was assessed with a titration of physiological concentrations (25, 100) and saturating for oxidant generation (500 μM). All protocols were repeated without creatine in the assay buffer to permit comparisons with the creatine condition, thereby allowing assessments of mitochondrial creatine sensitivity. Bundles were lyophilized in a freeze-dryer (Labconco, Kansas City, MO, USA) for >4 h and weighed on a microbalance (Sartorius Cubis Microbalance, Gottingen, Germany). The rate of mH₂O₂ emission was calculated using a standard curve under the same assay conditions and then normalized to fibre bundle dry weight.

### Mitochondrial Calcium Retention Capacity

This assay was measured spectrofluorometrically (QuantaMaster 80, HORIBA Scientific) in a cuvette with 300 μL assay buffer containing 1 μM Calcium Green-5N (Invitrogen), 2 μM thapsigargin, 5 μM blebbistatin, and 40 μM EGTA while maintained at 37° C. with continuous stirring. 5 mM glutamate, 2 mM malate, 5 mM ADP and 20 mM creatine were added to the assay buffer and minimum fluorescence was recorded. 4 nmol pulses of CaCl₂ were added until the mitochondrial permeability transition pore (mPTP) opening was observed as an increase in fluorescence corresponding to net mitochondrial calcium release, at which point saturating pulses of CaCl₂ were used to establish maximum fluorescence. Changes in free Ca²⁺ during mitochondrial Ca²⁺ uptake were then calculated using the known K_{d} for Calcium Green-5N and equations established for calculating free ion concentration. Calcium retention was then normalized to fibre bundle dry weight.

### Histochemical & Immunofluorescent Staining

Samples collected were cut into 8 82 m thick serial cross sections with a cryostat (Thermo Fisher Scientific; Kalamazoo, MI, United States;) maintained at −20° C. on Fisherbrand Superfrost Plus slides (Thermo Fisher Scientific). Hematoxylin and eosin (H&E) and Masson's trichrome staining was used to assess muscle health and fibrosis. Centralized nuclei, a marker of regenerating fibres, was analyzed by selecting five evenly spaced images throughout the muscle and averaging the result. Areas of damage (fibres with fragmented sarcoplasm and immune cell infiltration) were expressed as a percentage of the entire muscle section. Images were taken using EVOS M7000 imager (Thermo Fisher Scientific) using 20× magnification and analyzed using ImageJ (http://imagej.nih.gov/ij/). To determine fibrotic area, skeletal muscle sections were stained with Masson's Trichrome (fibrotic regions stain blue for collagen) and expressed as a percentage of a region of interest, which included the entire muscle section with any artifacts removed. Sections were imaged Nikon 90i-eclipse microscope (Nikon Inc., Melville, NY) and analysed with the NIS Elements AR software (v4.6, Nikon). Immunofluorescent analysis of myosin heavy chain expression was previously described. Images were taken with EVOS M7000 equipped with standard red, green, blue filters cubes. Fibres that were not positively stained were considered IIX fibres. These images were then analyzed with ImageJ for minimal Feret's diameter as an index of muscle atrophy.

### Western Blotting

Frozen sections of quadriceps and diaphragm from each muscle (approximately 10-30 mg in size) were homogenized using a plastic microcentrifuge tube with tapered Teflon pestle in ice-cold lysis buffer containing (in mM) (20 Tris/HCl, 150 NaCl, 1 EDTA, 1 EGTA, 1% Triton X-100, 2.5 Na₄O₇P₂, 1 Na₃VO₄, pH 7.0) supplemented with protease (Sigma) and phosphatase inhibitors (Roche). A 12% gel was used to separate electron transport chain proteins and all other proteins were separated on a 10% gel. Following, all gels were transferred onto 0.2μm low fluorescence PVDF membrane (Bio-Rad, Mississauga, Canada) and blocked with Li-COR Intercept Blocking Buffer (LI-COR, Lincoln NE, USA) for 1 hour. Membranes were then incubated with specific primary antibodies (listed below) overnight at 4° C. A commercially available monoclonal rodent OXPHOS Cocktail (ab110413; Abcam, Cambridge, UK, 1:250 dilution) including V-ATP5A (55 kDa), III-UQCRC2 (48 kDa), IV-MTCO1 (40 kDa), II-SDHB (30 kDa), and I-NDUFB8 (20 kDa) were used to detect electron transport chain proteins. Commercially available polyclonal antibodies were used to detect p-AMPKα (Thr172; 62kDa) (2535, Cell Signaling Technologies (CST), 1:1000), AMPKα (62 kDa; 2532, CST, 1:500), p-p-38MAPK (Thr180/Tyr182;43 kDa;) (9211, CST, 1:1000) and p38MAPK (40 kDa; 9212, CST, 1:500). After overnight primary antibody incubation, membranes were washed three times (5 minutes each time) in TBS-T and incubated at room temperature with appropriate fluorescent secondary antibody (LI-COR). Prior to detection, membranes were washed in TBS-T three times for 5 minutes and then imaged using infrared imaging (LI-COR CLx; LI-COR, Lincoln, NE, USA) and quantified by densitometry (ImageJ, http://imagej.nih.gov/ij/). All images were normalized total protein from the same membrane stained using Amido Black total protein-stained membrane (A8181, Sigma).

### Functional Assays and Serum Creatine Kinase

Voluntary wheel running, cage hang time and forelimb grip strength were assessed two days before sacrifice. Serum derived from cardiac puncture at the time of sacrifice were assessed spectrofluorometrically for creatine kinase activity (U/L).

### Results

### ALY688 Treatment Attenuates Fibrosis in the Diaphragm of D2.mdx Mice

The effect of ALY688 treatment was examined on diaphragm fibrosis defined as the absolute increase in collagen relative to levels observed in wildtype (WT). Using Masson's trichrome staining (FIG. 1B), a 5.5-fold increase in collagen content was observed in diaphragm from vehicle treated D2.mdx mice indicating fibrosis is present at a very early stage of disease development (VEH vs WT, FIG. 1B). The results showed that both doses of ALY688 attenuated this fibrosis by 66.7-67.6%. Collagen content increased from 0.5% to 1.1% in tibialis anterior in D2.mdx-VEH mice compared to WT (FIG. 1C) which indicates the diaphragm is more severely affected at this young age. Collectively, these findings indicate that early treatment with ALY688 decreases fibrosis in muscles severely affected by dystrophin mutation.

### ALY688 Attenuates Atrophy of D2.mdx Diaphragm in a Fibre Type-specific Manner

Minimal feret diameter was also examined in each fiber type as a measure of muscle atrophy. The high dose of ALY688 completely prevented atrophy in MHC I fibers. Additionally, in IIA fibers, minimal feret diameter was reduced from 24 μm in WT to 20 μm in VEH compared to 22 μm in HD (FIG. 2A). In IIB fibers, minimal feret diameter was reduced from 26 μm in WT to 23 μm in VEH compared to 24 μm in HD. A similar prevention of atrophy in IIB fibers was seen in LD.

There were no differences in minimal feret diameter in any fiber type of tibialis anterior between the groups (FIG. 2B). This finding suggests that the modest fibrosis in tibialis anterior seen at a young age (FIG. 1C) precedes atrophy given previous studies have shown reductions in fiber size in this muscle at later stages of disease in mdx mice.

### ALY688 Treatment Alters Markers of Inflammation

The effect of ALY688 was examined on markers of muscle damage and inflammation in the diaphragm and tibialis anterior. H&E staining was used to assess the degree of damaged areas consisting of areas of necrosis that often correspond to fibrosis (FIG. 2A and FIG. 10A). All D2.mdx groups demonstrated an increase in damaged areas (3.7-to-3.9-fold change vs WT; FIG. 3A) suggesting the attenuated fibrosis (FIG. 1B) with the drug occurred independently of generalized fiber damage. Next, inflammation was assessed only with high dose ALY688 given that both doses were equally as effective in attenuating fibrosis but the high dose ALY688 was more consistent in preventing atrophy. IL-6 mRNA content was increased in diaphragm from D2.mdx-VEH mice which was attenuated by the high dose (51.8% increase vs WT; FIG. 3B). In contrast, ALY688 increased IL-6 protein content in the diaphragm (69.1% increase vs VEH; FIG. 3C). This result in muscle tissue lysate does not rule out the possibility that expression of IL-6 in non-muscle cell types occurred (see Discussion). Increases in IL-1β mRNA in D2.mdx-VEH mice (2.7-to-3.0-fold increase vs WT) were not affected by the drug (FIG. 3B) while protein content was similar between all groups (FIG. 3C). TNF-α and IL-10 mRNA and protein contents were not different between groups. TGF-β protein content was increased in response to the drug (20.3% increase vs VEH; FIG. 3C) although mRNA contents were unchanged in all groups (FIG. 3B).

In the tibialis anterior, robust increases in damaged areas were noted in all three D2.mdx groups (17.2-to-22.6-fold increase vs WT; FIG. 10A) in contrast to the small degree of fibrosis seen in FIG. 1A-1C. D2.mdx-VEH mice demonstrated greater mRNA contents of TNF-α, IL-1β, IL-6, IL-10 and TGF-β which were paralleled by greater protein contents for TGF-β (9.7-fold increase vs VEH; FIGS. 11B, 11 C) compared to wildtype. The high dose of ALY688 attenuated TNF-α mRNA compared to VEH (41.4%; FIG. 10B). TNF-α protein content was not detectable in wildtype but increased to 40.6 pg/ml in VEH. HD prevented 83.9% of this increase (FIG. 10C).

ALY688 Lowers Mitochondrial H₂O₂ Emission by Enhancing ADP Responsiveness

Mitochondria produce H₂O₂ during oxidative phosphorylation of ADP to ATP. As cellular consumption of ATP increases, the cycling of ADP to the matrix is accelerated. Increasing matrix ADP attenuates membrane potential which in turn lowers mH₂O₂ emission given the two latter processes are inversely proportional. Given these principles, in vitro assay conditions were designed to capture the effect of increasing metabolic demand on oxidant generation by titrating ADP following induction of mH₂O₂ emission from mitochondria with NADH-generating substrates (pyruvate/malate) to support Complex I (FIG. 4A). This approach revealed that mH₂O₂ emission was higher at each ADP concentration in D2.mdx diaphragm and quadriceps (2.0-to-9.1-fold increase vs WT; FIGS. 4B, 4C). This elevation was due specifically to a reduced ability of ADP to attenuate mH₂O₂ relative to wildtype muscle. The results suggest that dystrophin mutation leads to a greater degree of mH₂O₂ emission across a range of oxidative phosphorylation kinetics. The high dose of ALY688 completely preserved mitochondrial responsiveness to ADP and restored mH₂O₂ emission to normal kinetics in diaphragm (8.7 to 70.7%; FIG. 4B). In quadriceps, both doses of the drug partially attenuated mH₂O₂ emission (45.9 to 62.3%; FIG. 4C). mH₂O₂ emission was not assessed in tibialis anterior.

In separate in vitro experiments, succinate (FADH₂; Complex II) was used to stimulate oxidant generation at Complex I as occurs through a unique reverse electron flow from Complex II to Complex. Furthermore, these kinetics in both the absence and presence of creatine given creatine accelerates matrix ADP/ATP cycling which can alter the influence of ADP in attenuating mH₂O₂, at least in response to succinate. Using this approach, it was found that succinate-induced mH₂O₂ emission was also elevated in D2.mdx diaphragm but there was no effect of either drug dose (1.1-to-2.3-fold increase vs WT; FIG. 11A, 11C). However, increased mH₂O₂ emission seen in quadriceps from D2.mdx mice were robustly attenuated by both doses (11.3 to 73.6%; FIGS. 11B, 11D).

Thus, while high doses lowered mitochondrial H₂O₂ emission due to forward electron flow from Complex I and supported by glucose-derived substrate (pyruvate) in both muscles, succinate-induced mH₂O₂ emission by reverse electron flow to Complex I was attenuated only in the quadriceps. These findings highlight the complexity of mitochondrial redox regulation across muscle types. The overall findings demonstrate that ALY688 lowers mH₂O₂ by preserving mitochondrial responsiveness to ADP.

Mitochondrial respiration was also examined as an index of oxidative phosphorylation in both quadriceps and diaphragm. D2.mdx mice demonstrated reduced ADP-stimulated respiration in multiple substrate conditions with generally no effect of either dose in both muscles (−25.9 to −67.0% vs WT; FIGS. 5A, 5B; FIGS. 12A-12D). Assessments of mitochondrial content markers through western blotting of electron transport chain complexes subunits were also completed in both muscles (FIGS. 5C, 5D). Overall, specific subunits of complex III and V, were significantly lowered in disease state groups in both the quadriceps and diaphragm (−14.7 to −31.5% vs WT) and total sum of complexes were reduced in diaphragm (−13.8 to −19.3% vs WT). HD treatment significantly reduced total sum in quadriceps only (−29.1% vs WT) while LD treatment significantly lowered complex IV content in the diaphragm (−27.0% vs WT).

In addition to mH₂O₂ emission and respiration, the potential for calcium-induced mitochondrial induction of apoptosis was also assessed. However, there was no differences between groups when assessing mitochondrial calcium retention capacity as a marker of permeability transition pore formation. Collectively, these mitochondrial assessments point to a more specific relationship between muscle fibrosis, atrophy and mitochondrial redox stress underlying the effects of ALY688 on diaphragm in D2.mdx mice.

### ALY688 Remodels Muscle Force Production

It was next determined whether muscle force production was different between groups. As expected, D2.mdx mice demonstrated lower muscle force production in both diaphragm strips and quadriceps (FIGS. 6A, 6B). Both doses of ALY688 lowered force relative to both wildtype and D2.mdx in diaphragm (−2.3% to −43.0% vs VEH & WT) but had no effect on quadriceps. There was less recovery from fatigue in diaphragm with the high dose and in quadriceps with the low dose (−5.3% to −50.9%). While initially unexpected, these assessments did not translate to attenuated physical activity behaviors as noted by similar grip strength, cage hang time and voluntary wheel running between all D2.mdx groups (−15.1% to −91.0% vs WT FIGS. 6C-6E) or serum creatine kinase (marker of muscle damage) in the high dose group despite increases seen with the low dose (3.1-to-5.2-fold increase vs WT; FIG. 6F).

Collectively, these results suggest that the ability of ALY688 to attenuate both fibrosis and atrophy in diaphragm are not paralleled by increased force production.

### Discussion

Here, it is confirmed the adiponectin receptor agonist ALY688 has an anti-fibrotic that reduced collagen content in the diaphragm by 66.7-67.6% of very young D2.mdx mice after 3 weeks of daily treatment. ALY688 also partially attenuated diaphragm atrophy in multiple fiber types. These effects were associated with lower diaphragm mitochondrial reactive oxygen species which suggests a relationship exists between mitochondrial stress and myopathy in this model.

The partial prevention of fibrosis and atrophy in the diaphragm was not observed in the tibialis anterior. However, this is likely due to the minimal disease effects noted in this muscle at this young age demonstrating a heterogeneous response of muscle at this early stage of disease progression in the D2.mdx mouse. Specifically, when comparing D2.mdx vehicle-treated mice to wildtype, collagen content was many-fold higher in diaphragm but only two-fold higher in tibialis anterior. The results indicate that ALY688 is effective at partially preventing fibrosis and atrophy when a robust disease state is present in muscle (e.g. diaphragm). Future studies could consider longer term treatment effects of ALY688 in muscles that develop more robust fibrosis and atrophy.

In tibialis anterior, ALY688 attenuated TNF-α mRNA and protein suggesting a possible direct effect in fibers of this muscle. As there was little fibrosis and no apparent atrophy in this muscle, these results may add further credence to determining whether such early effects of treatment translate into longer-term prevention of the eventual pathology in D2.mdx mice that likely manifests in other muscles at later stages of disease progression.

Mitochondrial H₂O₂ emission in diaphragm muscle fibers was reduced with ALY688 treatment. This assessment was performed with NADH-generating substrates which specifically target Complex I of the electron transport chain.

### Example 2: Mitigation of Chamber-Specific Cardiac Fibrosis and Alternation of Mitochondrial Bioenergetics in an Early Onset Duchenne Muscular Dystrophy Mouse Model

The objective of this study is to determine the degree to which adiponectin-receptor agonism in 4-week-old D2.B10-DMD^{mdx}/2J (D2.mdx) mice influences chamber-specific cardiac fibrosis and early fibrotic signaling, specifically in correspondence with altered mitochondrial bioenergetics.

D2.mdx mice were treated daily subcutaneously beginning at 7 days of age for 3 weeks at 1 μL/g with ALY688 at 15 mg/kg body weight (high dose drug; HD) or with a saline (VEH) control. Mice were compared to age-matched wildtypes (DBA/2J). Histopathological assessments identified significant elevations to left atrial and right ventricular (RV) fibrosis in VEH mice (+385%), which were both completely rescued by HD. As no fibrosis was observed in LV, this chamber served as a comparison to RV for exploring underlying mitochondrial relationships to fibrosis. In RV, mH₂O₂ emission assessed in the presence of a range of [ADP] was increased in VEH (+88%) vs wildtype but did not change with HD when stimulated by pyruvate (complex I substrate). ADP-stimulated respiration supported by pyruvate was lower in VEH (−49%) vs wildtype but completely rescued by HD. In LV, mH₂O₂ emissions supported by pyruvate were not different between groups. However, ADP-stimulated respiration supported by pyruvate was increased in VEH (36%) and HD (46%) vs wildtype whereas HD had no effect vs VEH.

### Example 3: Study of Effects of ALY688 on Memory Impairment in the D2.mdx Mouse Model of Duchenne Muscular Dystrophy

### Methods & Materials

### Animals

Male D2.mdx mice originated from a colony maintained at York University (Toronto, Canada) and sourced from Jackson Laboratories (Stock Number 013141, Bar Harbor, United States). Mice were treated daily from days 7 to 28 of age with ALY688 at 15 mg/kg b.w. (D2.mdx-ALY688) or saline control (D2.mdx-VEH). This age was chosen given it captures an early stage of disease progression in cardiac and skeletal muscle. Mice were sacrificed 20-24 hr after the last dose. Breeding of wildtype mice was unsuccessful. Instead, three-week-old wildtype DBA/2J WT mice (D2A) were obtained from Jackson Laboratories (Stock number 000671, Bar Harbor, United States) and allowed to acclimatize for one week before sacrifice. Mice were housed in standard 12:12-hr light:dark cycles and were allowed access to standard rodent chow and water ad libitum. 4 days prior to sacrifice, all groups underwent novel object recognition acclimatization and testing as described below. Mice were anesthetized under 5% isoflurane vaporized in medical air (21% oxygen) at a flow rate of 2 L/min and then maintained at 2-3% prior to euthanasia by exsanguination. Muscles were removed and used for another investigation (under review at the time of this submission). The right and left hippocampus were then quickly dissected from the brain with a portion placed immediately into ice-cold BIOPS, containing (in mM): 50 MES Hydrate, 7.23 K₂EGTA, 2.77 CaK₂EGTA, 20 imidazole, 0.5 dithiothreitol, 20 taurine, 5.77 ATP, 15 PCr, and 6.56 MgCl₂·6H₂O (pH 7.1) or flash-frozen in liquid nitrogen and stored at −80° C. for RNA isolation and western blotting. All experiments and procedures were approved by the Animal Care Committee at York University (AUP Approval Number 2016-18) in accordance with the Canadian Council on Animal Care.

### Novel Object Recognition Test

The novel object recognition test (NOR) was performed as previously described in literature. Briefly, animals were placed in an open field area (40 cm×40 cm×40 cm). Sessions were recorded with a cell-phone video camera secured above the apparatus. Testing was performed in 3 stages: acclimation, habituation and testing. During acclimation, mice were placed into the arena and allowed to freely move for 5 minutes each day for 4 days preceding testing. The following day, habituation was performed, where two identical objects (Object 1) were placed in opposite corners of the arena and mice were left to explore for 10 minutes. Objects were similar in size to the animals and were chosen to ensure novelty in all trials. Following a habituation period, mice were returned to a neutral cage for 30 minutes. Thereafter, mice were placed back in the arena with one familiar object and one novel object (Object 2) and allowed to explore for 10 minutes. Object exploration time was defined as the time the mouse interacted with the object, defined by sniffing or touching the object when the mouse is less than 2 cm from the object. Sitting or standing on the object was not included unless the mouse sniffed the object while climbing on it. For the trial to be considered the animal must have interacted with the object for greater than 20 seconds. Discrimination ratio (DR) was calculated as follows: DR=(Time(object 2)−Time(object 1))/Total time.

### High-Resolution Respirometry

Mitochondrial oxygen consumption (respiration) was measured using the in situ brain permeabilization. Following weighing, a portion of the hippocampus samples were quickly minced with scissors in a chilled tube containing BIOPS buffer followed by immediate placement into an Oxygraph-2K respirometer (Oroboros Instruments, Austria) containing MiR05 respiration medium (0.5 mM EGTA, 3 mM MgCl₂, 10 mM KH₂PO₄, 20 mM HEPES, 60 mM K-lactobionate, 110 mM sucrose, 1 g/L BSA; pH 7.2) at 37° C. with constant stirring at 750 rpm. Samples were equilibrated in respiration buffer for 10 minutes before the addition of 50 μg/mL saponin to facilitate permeabilization of the tissue. Following permeabilization, pyruvate-stimulated respiration was examined in the brain using 5 mM pyruvate & 2 mM malate to generate NADH and saturate electron entry into Complex I. To examine State III respiration as an index of oxidative phosphorylation, ADP was then added at a concentration of 15 uM to approximate the concentrations reported in human brains using non-invasive MRS assessments. Cytochrome c was added as a test of mitochondrial outer membrane integrity. All experiments demonstrated <10% increase in respiration. Each protocol was initiated with a starting [O₂] of ˜350 μM and was completed before the oxygraph chamber [O₂] reached 150 μM. Polarographic oxygen measurements were acquired in 2 s intervals with the rate of respiration derived from 40 data points and expressed as pmol/s/mg wet weight. Chemicals and reagents were purchased from Sigma (St. Louis, Mo., USA), BioShop (Burlington, ONT, Canada).

### RNA Isolation & Quantitative PCR

Total RNA was isolated from hippocampus using the Aurum Total RNA Mini Kit (Bio-Rad; Mississauga, ON, Canada) according to manufacturer's instructions, and reverse transcribed into cDNA by M-MLV reverse transcriptase and oligo (dT) primers (Qiagen). cDNA was then amplified in a CFX384 Touch Real-Time PCR Detection Systems (Bio-Rad) with a SYBR Green master mix and specific primers. Gene expression was normalized to a Rplp0 control and relative differences were determined using the ΔΔCt method (Livak, K. J., & Schmittgen, T. D. (2001). Analysis of relative gene expression data using real-time quantitative PCR and the 2(-Delta Delta C(T)) Method. Methods, 25 (4), 402-408. doi: 10.1006/meth.2001.1262), normalized to D2A expression. The primers used to probe for mouse cytokines are as follows: IL-1β Forward 5′-GCAGCACATCAACAAGAG-3′ (SEQ ID NO: 1), Reverse 5′-AGCAGGTTATCATCATCATC-3′ (SEQ ID NO: 2); TNF-αForward 5′-AGAATGAGGCTGGATAAGAT-3′ (SEQ ID NO: 3), Reverse 5′-GAGGCAACAAGGTAGAGA-3′ (SEQ ID NO: 4); IL-6 Forward 5′-ACAGAAGGAGTGGCTAAG-3′ (SEQ ID NO: 5), Reverse 5′-AGAGAACAACATAAGTCAGATAC-3′ (SEQ ID NO: 6); IL-10 Forward 5′-ATAACTGCACCCACTTCCCA-3′ (SEQ ID NO: 7), Reverse 5′-GGGCATCACTTCTACCAGGT-3′ (SEQ ID NO: 8); Rplp0 Forward 5′-TTGGAGTGACATCGTCTT-3′ (SEQ ID NO: 9), Reverse 5′-ATCTTGAGGAAGTAGTTGGA-3′ (SEQ ID NO: 10).

### Cytokine Profiling

Protein contents of TGF-β, TNF-α, IL-1β, IL-6 and IL-10 in serum were measured by flow cytometry using LEGENDplex™ Mouse Custom Panel (BioLegend; San Diego, CA, USA). Serum, collected from all groups, was diluted four-fold in the assay buffer. Assay was completed according to the manufacturer's instructions on the Attune NxT flow cytometer (Thermo Fisher). The FCS files generated on the flow cytometer were analyzed using the LEGENDplex™ cloud-based analysis software.

### Western Blotting

Hippocampus tissue (collected from the same mice utilized in high-resolution respirometry experiments) was homogenized in cold lysis buffer containing (in mM; 20 Tris/HCl, 150 NaCl, 1 EDTA, 1 EGTA, 1% Triton X-100, 2.5 Na₄O₇P₂, 1 Na₃VO₄, pH 7.0) supplemented with protease and phosphatase inhibitors (Sigma) according to the protocol established in Hughes MC et al. “Early myopathy in Duchenne muscular dystrophy is associated with elevated mitochondrial H2O2 emission during impaired oxidative phosphorylation.” Journal of Cachexia, Sarcopenia and Muscle. 2019; 10:643-61. Detection of electron transport chain complex subunits were performed according to the procedure in Hughes, M. C., et al. (2019), “Early myopathy in Duchenne muscular dystrophy is associated with elevated mitochondrial H2O2 emission during impaired oxidative phosphorylation.” Journal of Cachexia, Sarcopenia and Muscle, 10 (3), 643-661. For detection of total AMPK, p-AMPK, p-ULK1, p62, and LC3BII/I, proteins were transferred to a 0.2 μm low fluorescence polyvinylidene difluoride membrane using a Bio-Rad Trans Blot Turbo. Membranes were then blocked in Licor Intercept Blocking buffer diluted 1:1 in PBS. Membranes were then incubated with appropriate primaries diluted in blocking buffer (LI-COR, Lincoln NE, USA) as follows: p-AMPK (1:1000; t192 CST 2535), total AMPK (1:500; CST 2532), p-ULK1 (ser757, the target of Raptor; 1:500; CST14202), p62 (1:1000; CST 5114) and LC3B (1:1000; CST 2775) overnight at 4° C. Membranes were then washed three times for 5 mins in TBS-T and incubated at room temperature with appropriate fluorescent secondary antibody (LI-COR). The same washing protocol was repeated and then membranes were detected using infrared imaging (LI-COR CLx; LI-COR, Lincoln, NE, USA) and quantified by densitometry (ImageJ, http://imagej.nih.gov/ij/). All images were normalized to total protein from the same membrane stained using Amido Black total protein stain (A8181, Sigma).

All remaining proteins were detected on 0.45 μm nitrocellulose and blocked with 5% nonfat dry milk in TBS-T. Membranes were incubated with the appropriate primary antibodies as follows: APP (BioLegend 825001), soluble APPα (IBL 11088), soluble APPβ (BioLegend 813401), BACE1 (CST 5606), ADAM10 (Abcam ab1997), p-tau (serine 202; CST 11834), total tau (CST 4019), p-Raptor (serine 792; CST 2083), total Raptor (CST 2280), p-p70s6K (thr389; CST 9206S), total p70s6k (Santa Cruz SC-230), NeuN (CST 24307) and pro-BDNF (SC SC-65514) diluted to 1:1000 in 5% BSA overnight at 4° C. Following primary antibody incubation and washing, membranes were incubated in horseradish peroxidase secondary antibodies diluted 1:5000 in 1% nonfat dry milk-TBS-T. Membranes were washed (3×5 min in TBST) and protein bands were imaged using enhanced chemiluminescence (Western lightning Plus-ELC; PerkinElmer, 105001EA) and ChemiDoc Imaging System (Bio-Rad). All proteins were normalized to total protein obtained from ponceau stain. Images were analyzed via Alpha View Software (ProteinSimple).

### Statistics

Results are expressed as means±SD. The level of significance was set to p<0.05 for all statistics. D'Agostino-Pearson normality tests (GraphPad Prism Software, La Jolla, CA, USA) confirmed that all data were normally distributed, one-way ANOVAs were utilized with two-stage step-up method of Benjamini, Krieger and Yekutieli post-hoc analyses for False Discovery rate (FDR) corrections in multiple-group comparisons. All reported p-values are FDR-adjusted p values (traditionally termed ‘q’).

### Results

D2.mdx-VEH mice demonstrated impaired recognition memory as shown by a lower discrimination index in the NOR test (FIGS. 7A,7B). This decrement was completely prevented by ALY688 (FIGS. 7A, 7B). Hippocampal mitochondrial pyruvate-supported respiration stimulated by ADP at physiological concentrations (see methods) was lower in D2.mdx-VEH but completely preserved by ALY688 (FIG. 7C). These differences between groups likely reflect adaptive reprogramming intrinsic to mitochondria given electron transport chain subunit contents were similar in all groups (FIGS. 7D, 7E).

No significant changes were observed in hippocampal mRNA of IL-6, IL-162, TNF-α or IL-10 (FIG. 8A-8D). Cytokine protein contents were assessed in serum due to tissue limitations in the hippocampus. Serum IL-6 and TNF-α were significantly elevated in D2.mdx-VEH mice vs D2A controls (FIGS. 8E, 8G). ALY688 attenuated IL-6 (FIG. 8E) and increased IL-1β relative to D2.mdx-VEH while also increasing IL-10 vs wildtype. (FIGS. 8F, 8H).

As shown in FIGS. 9B,9C, total AMPK protein was lower following treatment with ALY688. The degree of activation (p-AMPK or p-AMPK/AMPK) was not altered. ALY688 also completely prevented the increases in protein contents of total tau (marker of neurofibrillary tangles) and total raptor (upstream regulator of tangles and autophagy) seen in D2.mdx vs WT. However, phosphorylation (both absolute and relative to total protein) of tau, Raptor (serine 792; an AMPK specific phosphorylation site (Gwinn et al., 2008)) were similar in all groups (FIGS. 9A-9C). Markers of amyloidogenesis were similar in all groups as were BDNF and NeuN (data not shown).

In summary, the results show that ALY688 improved recognition memory in young D2.mdx mice following a short-term 21-day treatment paradigm. This effect was associated with a complete restoration of hippocampal pyruvate-supported mitochondrial respiration (index of glucose oxidation) when stimulating ATP synthesis with an ADP concentration matching the level reported in vivo in human brain.

### FIGURE DESCRIPTIONS

FIGS. 1A-1C: Schematic of study design and quantification of interfibrillar collagen accumulation in D2.mdx mice treated with ALY688. A visual representation of study design is provided in (FIG. 1A). 7 day old D2.mdx mice received subcutaneous injections of ALY688 at a low dose (LD; 3 mg/kg body weight) or high dose (HD; 15 mg/kg body weight) or a saline control for 21 days. Healthy D2A mice (wild type (WT)) were used as age matched controls. At day 18 of treatment, all four groups underwent whole body functional assays to assess voluntary muscle function. After 21 days of injections, all groups were sacrificed, and tissue collected for the outlined measures. Fibrosis was evaluated in diaphragm (FIG. 1B) and tibialis anterior (FIG. 1C) using Masson's trichrome staining. Results represent mean±SD; n=7-15. All p values are FDR-adjusted by Benjamini, Krieger and Yekutieli post-hoc analyses. *p<0.05 WT vs D2.mdx-VEH; ‡p<0.05 D2.mdx-VEH vs D2.mdx-LD; § p<0.05 D2.mdx-VEH vs D2.mdx-HD. Representative images of diaphragm (FIG. 1B, magnification, ×20) and tibialis anterior (FIG. 1C, magnification, ×20). Scale bar=100 μm.

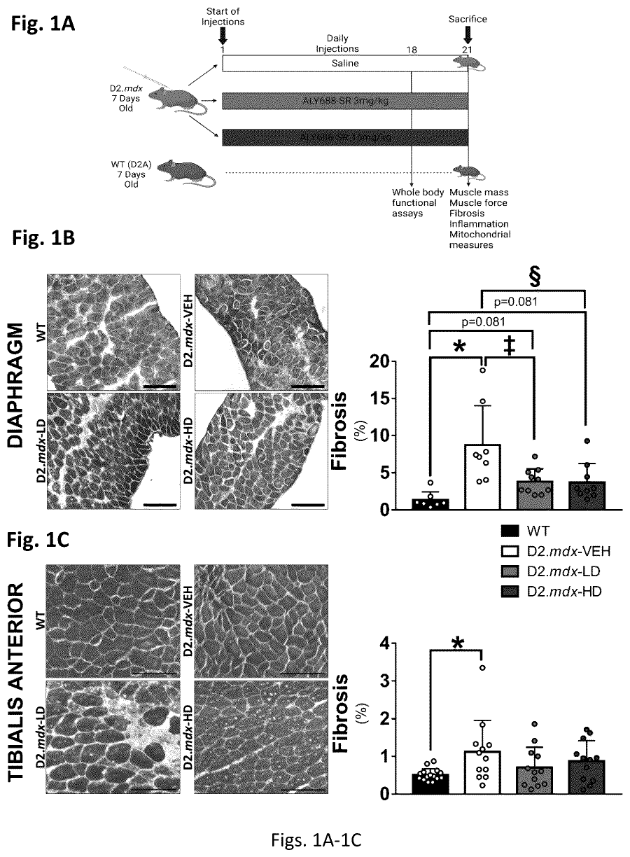

FIGS. 2A-2B: Assessments of fiber-type specific minimal feret diameter in D2.mdx mice treated with ALY688. Minimal feret diameter was assessed in diaphragm (FIG. 2A) and tibialis anterior (FIG. 2B) using immunofluorescent detection of myosin heavy chain isoforms. Results represent mean±SD; n=7-10. All p values are FDR-adjusted by Benjamini, Krieger and Yekuticli post-hoc analyses. *p<0.05 WT vs D2.mdx-VEH; #p<0.05 WT vs D2.mdx-LD; †p<0.05 WT vs D2.mdx-HD; ‡p<0.05 D2.mdx-VEH vs D2.mdx-LD; § p<0.05 D2.mdx-VEH vs D2.mdx-HD. Representative images of diaphragm (A, magnification, ×20) and tibialis anterior (C, magnification, ×20). Scale bar=100 μm.

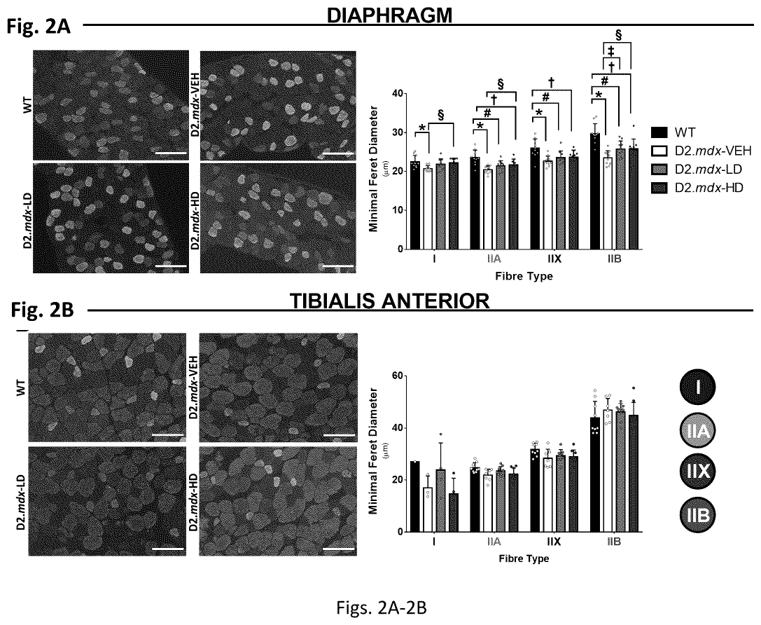

FIGS. 3A-3C: Effects of ALY688 on inflammation and muscle damage in D2.mdx diaphragm. As both doses of ALY688 were effective in reducing fibrosis in diaphragm, only high dose groups were examined in inflammation. (FIG. 3A) Hematoxilyn & eosin staining were used to assess areas of muscle damaged which includes areas of necrosis and fibrosis and expressed as a percentage of total area. Scale bar=100 μm. (FIG. 3B) qPCR were used to assess mRNA fold changes of IL-6, IL-1β, TNF-α, IL-10 and TGF-β across WT and D2.mdx mice treated with vehicle or high dose ALY688 treatment. qpCR results were normalized to Rplp00, and the subsequent ratios were presented as relative expression compared with WT values. (FIG. 3C) Protein levels of the same cytokines, were assessed by BioLegend Multiplex using flow cytometry. Results represent mean±SD; n=7-12. All p values are FDR-adjusted by Benjamini, Krieger and Yekutieli post-hoc analyses. *p<0.05 WT vs D2.mdx-VEH; #p<0.05 WT vs D2.mdx-LD; †p<0.05 WT vs D2.mdx-HD; § p<0.05 D2.mdx-VEH vs D2.mdx-HD.

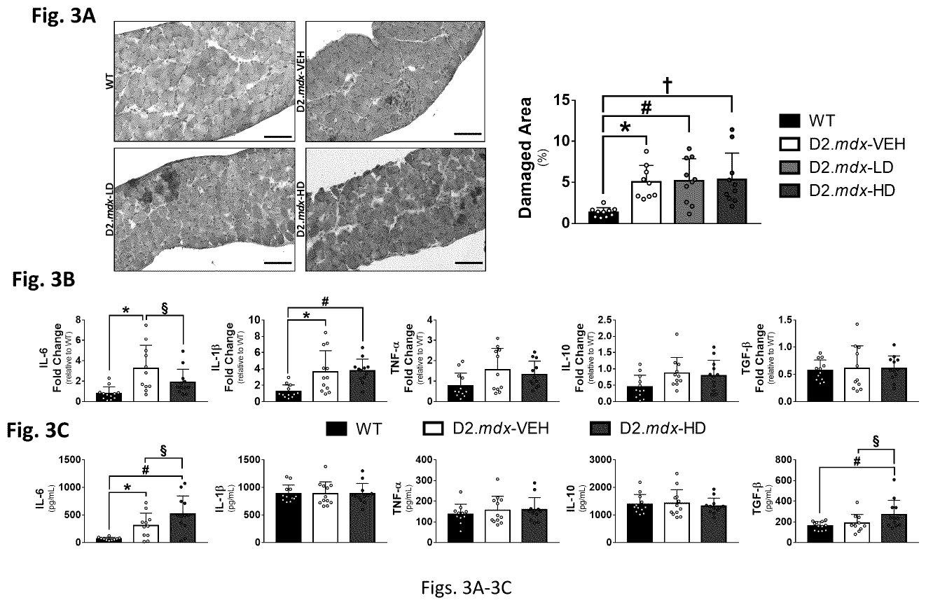

FIGS. 4A-4C: ALY688 enhances ADP-suppression of complex-I supported mitochondrial H₂O₂ emission. (FIG. 4A) Schematic representation of complex-I supported mitochondrial H₂O₂ emission (mH₂O₂) due to forward electron flow from NADH generated by pyruvate. In states of high membrane potential (low ADP), electrons at complex I may ‘slip’ prematurely at, for example, Complex I to produce superoxide radicals (O₂^{⋅-}) which are then converted to H₂O₂ by manganese superoxide dismutase (MnSOD). 10 mM pyruvate and 2 mM malate (to assist with continual flux through pyruvate dehydrogenase complex (PDC)), were used to stimulate electron flow through complex I in permeabilized fibre bundles from diaphragm (FIG. 4B) and quadriceps (FIG. 4C) in the absence of ADP (state II; high membrane potential). Subsequent titrations of physiological levels of ADP (‘resting muscle’, 25 uM; high periods of demand similar to intense exercise; 100 uM) and supramaximal (500 uM) attenuates mH₂O₂ by promoting forward electron flow and is expressed as a % of State II mH₂O₂ emission to reveal the degree of attenuation in response to ADP. Results represent mean±SD; n=10-12. All p values are FDR-adjusted by Benjamini, Krieger and Yekutieli post-hoc analyses. *p<0.05 WT vs D2.mdx-VEH; #p<0.05 WT vs D2.mdx-LD; †p<0.05 WT vs D2.mdx-HD; ‡p<0.05 D2.mdx-VEH vs D2.mdx-LD; § p<0.05 D2.mdx-VEH vs D2.mdx-HD; \hat{ }p<0.05 D2.mdx-LD vs D2.mdx-HD. TCA=tricarboxylic cycle; PDC=pyruvate dehydrogenase complex. O₂^{⋅-}=superoxide radicals.

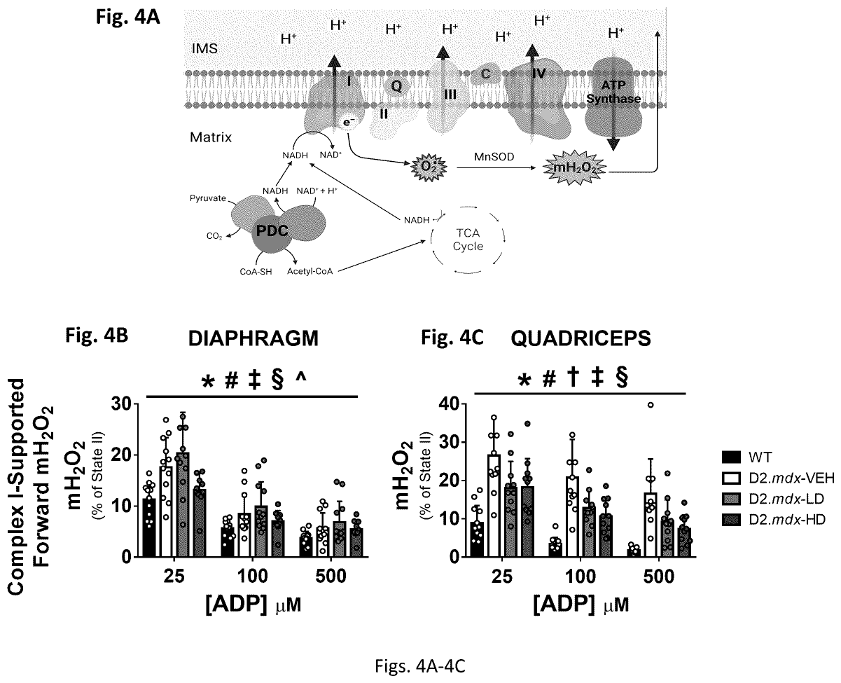

FIGS. 5A-5D: Reductions in pyruvate-supported mitochondrial respiration and protein markers of the electron transport chain in D2.mdx mice are not altered by ALY688. Complex-I supported respiration (assessed by oxygen flux, JO₂) stimulated by NADH generation through 5 mM pyruvate and 2 mM malate in permeabilized fibre across a range of ADP concentrations representing increasing states of metabolic demand: 25 μM (modeling resting muscle (30)); 100 μM (modeling high intensity exercise (51)), 500 uM (maximal) and 5000 μM (supramaximal) was assessed in the diaphragm (FIG. 5A) and quadriceps (FIG. 5B). Subunit of complexes I, II, III, IV and V were assessed by western blot in diaphragm (FIG. 5C) and quadriceps (FIG. 5D). Results represent mean±SD; n=9-12. All p values are FDR-adjusted by Benjamini, Krieger and Yekuticli post-hoc analyses. *p<0.05 WT vs D2.mdx-VEH; #p<0.05 WT vs D2.mdx-LD; †p<0.05 WT vs D2.mdx-HD.

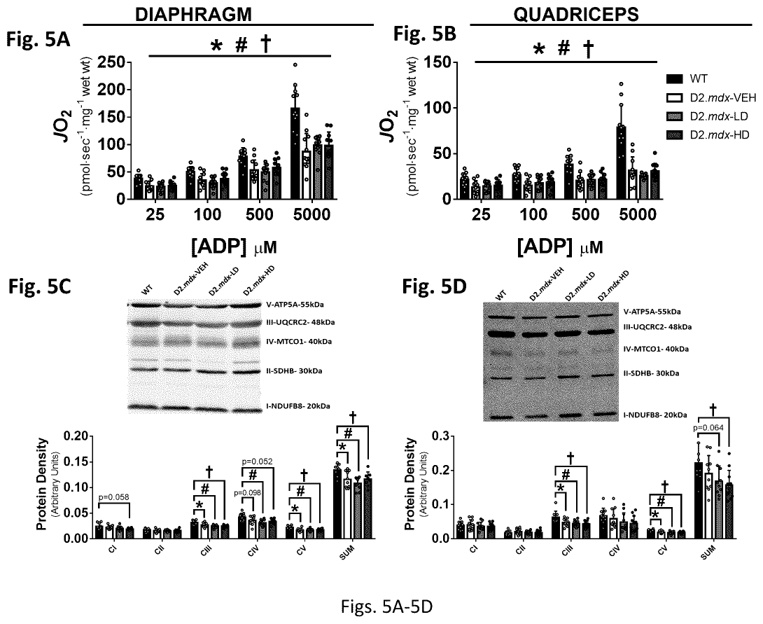

FIGS. 6A-6F: Muscle function assessments of D2.mdx treated with ALY688. Muscle force was assessed using force frequency protocols in in-vitro diaphragm strips (FIG. 6A) and in-situ quadriceps preparation (FIG. 6B). Serum creatine kinase was assessed as a clinical marker of muscle breakdown (FIG. 6C). Cage hang time (FIG. 6D), grip strength normalized to body weight (FIG. 6E), and distance travelled during 24 hours of voluntary wheel running (FIG. 6F) were assessed as measures of voluntary motor function. Results represent mean±SD; n=8-13 (FIGS. 6A, 6B); n=13-22 (C); n=21-26 (FIGS. 6D-6F). All p values are FDR-adjusted by Benjamini, Krieger and Yekutieli post-hoc analyses. *p<0.05 WT vs D2.mdx-VEH; #p<0.05 WT vs D2.mdx-LD; †p<0.05 WT vs D2.mdx-HD; ‡p<0.05 D2.mdx-VEH vs D2.mdx-LD; § p<0.05 D2.mdx-VEH vs D2.mdx-HD; \hat{ }p<0.05 D2.mdx-LD vs D2.mdx-HD.

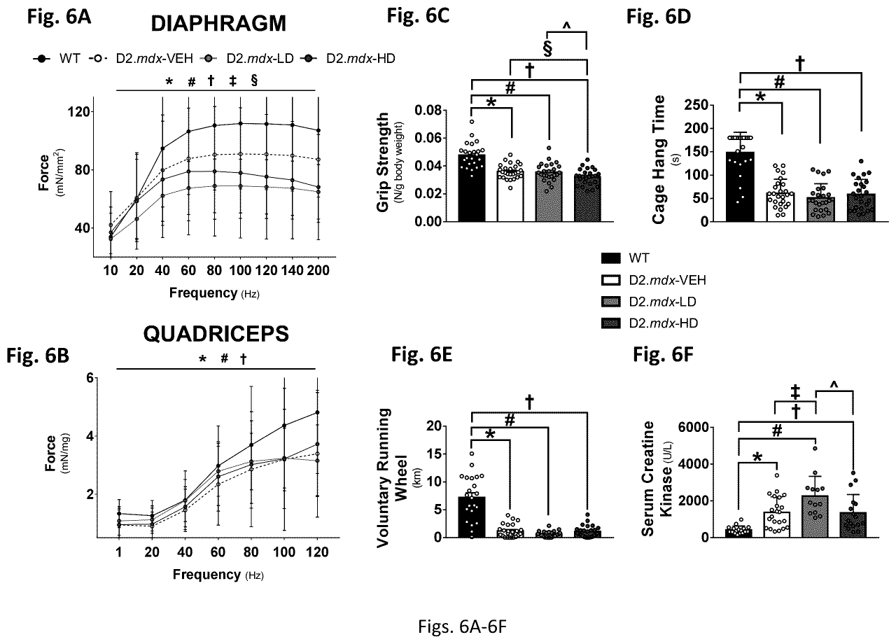

FIGS. 7A-7E: Novel object recognition (NOR) testing and hippocampal Complex I-supported respiration are preserved by ALY688 in 4-week-old D2.mdx mice. (FIG. 7A) Representative image of the NOR task with habituation (left), familiarization (middle) and testing (right). (FIG. 7B) Discrimination index was reduced in D2.mdx-VEH mice and was normalized with ALY688 treatment. (FIG. 7C) State III respiration was supported by Complex I substrates (NADH) pyruvate (5 mM) and malate (2 mM) and stimulated by a physiological concentration of ADP (15 μM). (FIGS. 7D, 7E) Protein content of electron transport chain components was quantified in the hippocampus. Data expressed as mean±SD with n=9-12 per group. *p≤0.05 WT vs D2.mdx-VEH; § p≤0.05 D2.mdx-VEH vs D2.mdx-ALY688.

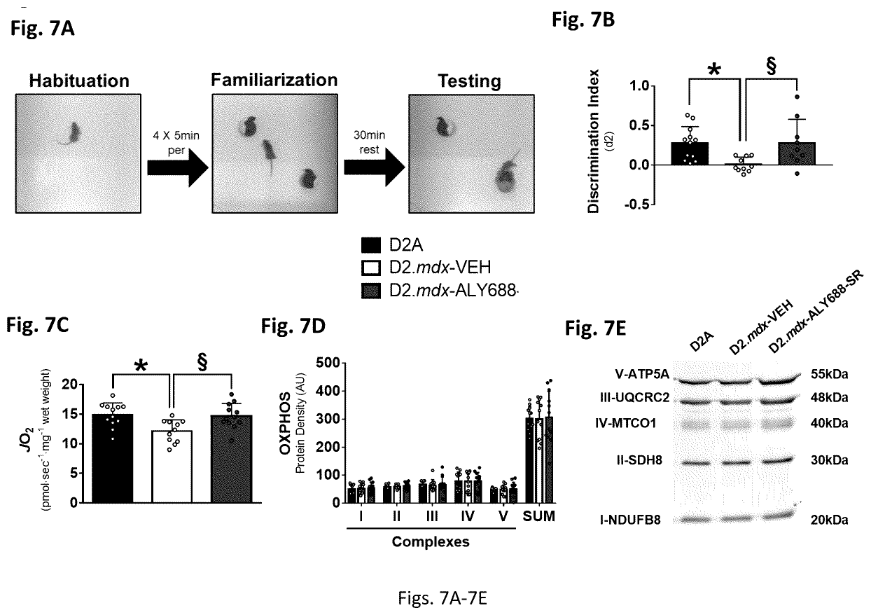

FIGS. 8A-8H: Hippocampus and serum inflammatory cytokine expression and contents. (FIGS. 8A-8D) Hippocampal mRNA fold changes of IL-6, IL-1β, TNF-α and IL-10 were expressed relative to D2A expression. (FIGS. 8E-8H) Serum levels of IL-6, IL-1β, TNF-α and IL-10 were quantified. Data expressed as mean±SD with n=7-12 per group. *p≤0.05 WT vs D2.mdx-VEH; #p≤0.05 WT vs D2.mdx-VEH; § p≤0.05 D2.mdx-VEH vs D2.mdx-ALY688. IL-6-interleukin-6, IL-1β-Interleukin-1 beta, TNF-α-tumor necrosis factor-alpha, IL-10-interleukin-10.

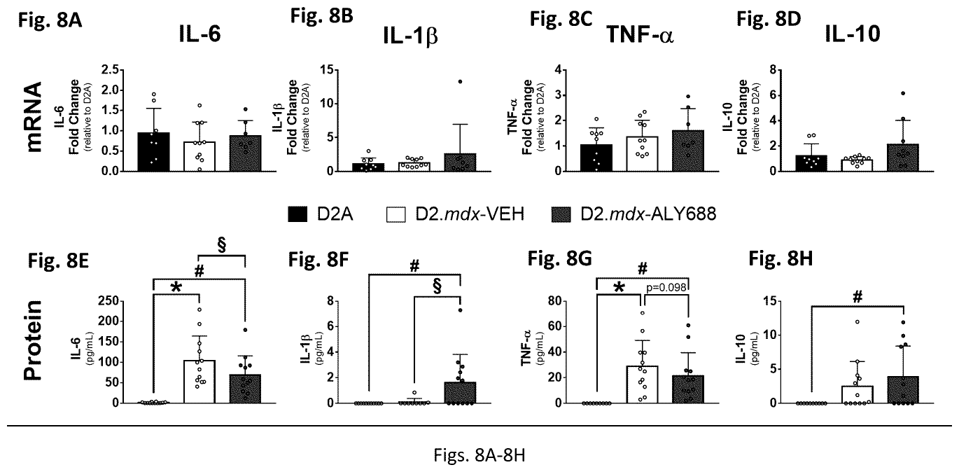

FIGS. 9A-9C: AMPK and downstream protein markers related to autophagy and neurofibrillary tangles. (FIG. 9A) Theoretical cascade linking AMPK—an adiponectin receptor target—to factors related to autophagy and neurofibrillary tangles. The AMPK target, Raptor is rendered inactive upon its phosphorylation, allowing for the induction of autophagy through the phosphorylation of ULK-1, allowing for clearance of dysfunctional mitochondria and proteins. Alternatively, activation of the mTORC1 complex leads to increased protein synthesis via p70s6k1 which has been implicated in tau phosphorylation, the destabilization of microtubules and the generation of neurofibrillary tangles (Iqbal et al., 2010; Pei et al., 2006). Made with BioRender. (FIGS. 9B, 9C) Western blot markers for targets outlined in assessed in hippocampus tissue (A). Data expressed as mean±SD with n=6-12 per group. *p≤0.05 WT vs D2.mdx-VEH; § p≤0.05 D2.mdx-VEH vs D2.mdx-ALY688. AMPK-AMP kinase, mTORC1-mammalian target of rapamycin complex 1, Raptor-Regulatory-associated protein of mTOR, ULKI-Unc-51like autophagy activating kinase, p70s6k1-Ribosomal protein S6 kinase β 1.

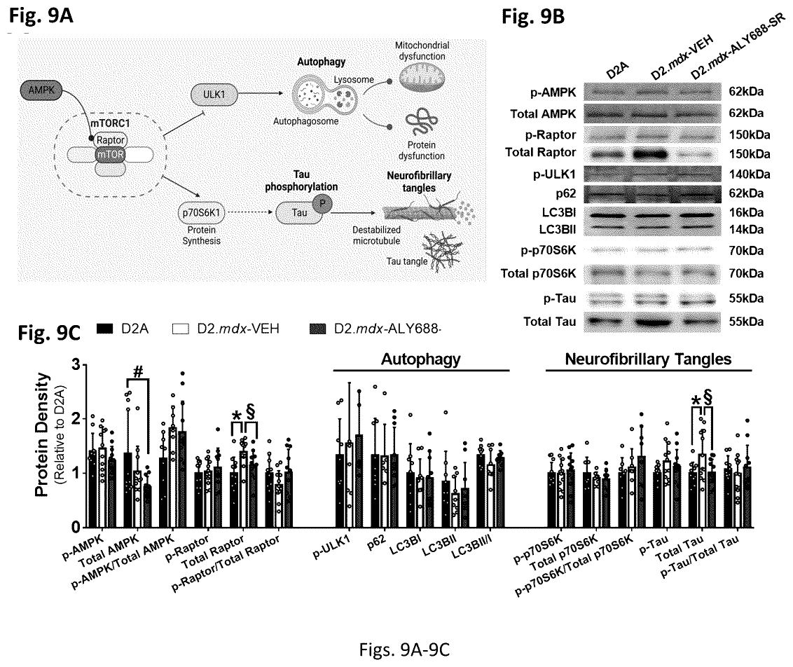

FIGS. 10A-10C: Effects of ALY688 on inflammation and muscle damage in D2.mdx tibialis anterior. (FIG. 10A) Hematoxylin & eosin staining were used to assess areas of muscle damaged which includes areas of necrosis and fibrosis and expressed as a percentage of total area. Scale bar=100 μm. (FIG. 10B) qPCR were used to assess mRNA fold changes of TNF-α, IL-1β, IL-6, IL-10 and TGF-β across WT and D2.mdx mice treated with vehicle or high dose ALY688 treatment. qPCR results were normalized to Rplp00, and the subsequent ratios were presented as relative expression compared with WT values. (FIG. 10C) Protein levels of the same cytokines, were assessed by BioLegend Multiplex using flow cytometry. Results represent mean±SD; n=7-12. All p values are FDR-adjusted by Benjamini, Krieger and Yekuticli post-hoc analyses. *p<0.05 WT vs D2.mdx-VEH; #p<0.05 WT vs D2.mdx-LD; †p<0.05 WT vs D2.mdx-HD; § p<0.05 D2.mdx-VEH vs D2.mdx-HD.

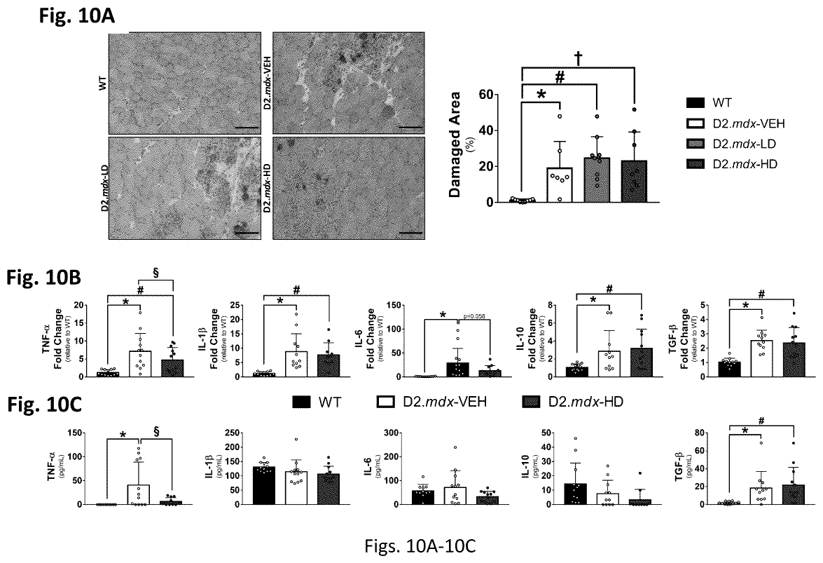

FIGS. 11A-11D: ALY688 enhances ADP attenuation of mitochondrial H₂O₂ emission supported by succinate in quadriceps. The ability of ADP to attenuate mitochondrial H₂O₂ emission (mH₂O₂) stimulated by succinate was assessed in permeabilized fibres of diaphragm (FIGS. 11A, 11B) and quadriceps (FIGS. 11C, 11D). 10 mM succinate (FADH₂) was used to stimulate electron flow in reverse direction from complex II to complex I to generate superoxide that is dismutated to H₂O₂. ADP's attenuation of mH₂O₂ was assessed in the absence (FIGS. 22A, 11C) and presence (FIGS. 11B, 11D) of creatine (accelerator of matrix ADP/ATP cycling) across a range of ADP concentrations depicting increasing metabolic demand. Analyses were performed in the diaphragm (A,B) and quadriceps (C,D). Results represent mean±SD; n=10-12. All p values are FDR-adjusted by Benjamini, Krieger and Yekutieli post-hoc analyses. *p<0.05 WT vs D2.mdx-VEH; #p<0.05 WT vs D2.mdx-LD; †p<0.05 WT vs D2.mdx-HD; ‡p<0.05 D2.mdx-VEH vs D2.mdx-LD; § p<0.05 D2.mdx-VEH vs D2.mdx-HD.

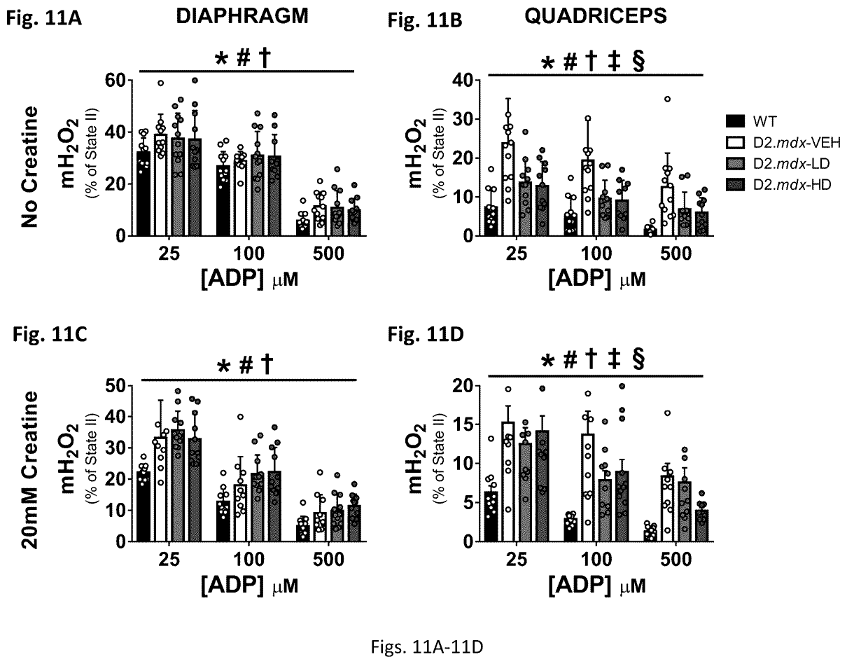

FIGS. 12A-12D: Mitochondrial respiration normalized to mitochondrial content markers is not altered by ALY688 treatment. Complex-I supported respiration (assessed by oxygen flux, JO₂) normalized to protein markers of the electron transport chain (from FIG. 6B) as an index of mitochondrial content in diaphragm (FIGS. 12A, 12B) and quadriceps (FIGS. 12C, 12D) in the absence (FIGS. 12A, 12C) and presence (FIGS. 12B, 12D) of 20 mM creatine (to accelerate ADP/ATP cycling) across a range of increasing metabolic demand (ADP concentrations). Results represent mean±SD; n=9-12. All p values are FDR-adjusted by Benjamini, Krieger and Yekutieli post-hoc analyses. *p<0.05 WT vs D2.mdx-VEH; #p<0.05 WT vs D2.mdx-LD; †p<0.05 WT vs D2.mdx-HD.

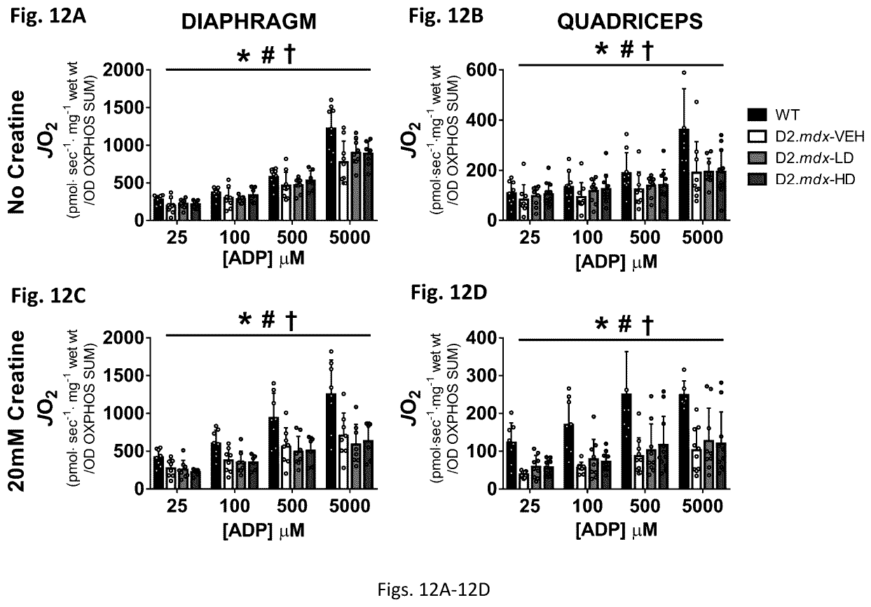

1. A method of treating a muscular dystrophinopathy, or a condition associated therewith, in a subject, comprising:
administering to the subject a therapeutically effective amount of a composition comprising ALY688.

2. The method of claim 1, wherein the muscular dystrophinopathy comprises Duchenne muscular dystrophy (DMD), Becker muscular dystrophy (BMD), DMD-associated dilated cardiomyopathy (DCM), facioscapulohumeral muscular dystrophy (FSHD), congenital muscular dystrophy (CMD), Emery-Dreiffus muscular dystrophy (EDMD), Limb-girdle muscular dystrophy (LGMD), myotonic dystrophy, and oculopharyngeal muscular dystrophy (OPMD).

3. The method of claim 1, wherein the administration comprises subcutaneous administration.

4. The method of claim 1, wherein the condition is damage and loss of function in the right ventricle.

5. The method of claim 1, wherein the condition is diaphragm atrophy and loss of function.

6. The method of claim 1, wherein the condition is memory impairment.

7. A method of treating memory or recognition impairment in a subject, comprising:
administering to the subject a therapeutically effective amount of a composition comprising ALY688.

8. The method of claim 7, wherein the administration comprises subcutaneous administration.

9. A method of ameliorating cardiac dysfunction associated with a muscular dystrophinopathy in a subject, comprising:
administering to the subject a therapeutically effective amount of a composition comprising ALY688.

10. A method of ameliorating respiratory dysfunction associated with a muscular dystrophinopathy in a subject, comprising:
administering to the subject a therapeutically effective amount of a composition comprising ALY688.


SEQUENCE LISTING (10 sequences)

SEQ ID NO: 1 - DNA, 18 bp, synthetic construct
  source 1..18: mol_type = other DNA
gcagcacatcaacaagag

SEQ ID NO: 2 - DNA, 20 bp, synthetic construct
  source 1..20: mol_type = other DNA
agcaggttatcatcatcatc

SEQ ID NO: 3 - DNA, 20 bp, synthetic construct
  source 1..20: mol_type = other DNA
agaatgaggctggataagat

SEQ ID NO: 4 - DNA, 18 bp, synthetic construct
  source 1..18: mol_type = other DNA
gaggcaacaaggtagaga

SEQ ID NO: 5 - DNA, 18 bp, synthetic construct
  source 1..18: mol_type = other DNA
acagaaggagtggctaag

SEQ ID NO: 6 - DNA, 23 bp, synthetic construct
  source 1..23: mol_type = other DNA
agagaacaacataagtcagatac

SEQ ID NO: 7 - DNA, 20 bp, synthetic construct
  source 1..20: mol_type = other DNA
ataactgcacccacttccca

SEQ ID NO: 8 - DNA, 20 bp, synthetic construct
  source 1..20: mol_type = other DNA
gggcatcacttctaccaggt

SEQ ID NO: 9 - DNA, 18 bp, synthetic construct
  source 1..18: mol_type = other DNA
ttggagtgacatcgtctt

SEQ ID NO: 10 - DNA, 20 bp, synthetic construct
  source 1..20: mol_type = other DNA
atcttgaggaagtagttgga